(19) United States
(12) Patent Application Publication
(10) Pub. No.: US 2009/0275845 A1
(43) Pub. Date: Nov. 5, 2009

(54) METHOD AND DEVICE FOR THE NON-INVASIVE DETECTION OF BLOOD FLOW AND ASSOCIATED PARAMETERS IN PARTICULAR ARTERIAL WAVEFORM AND BLOOD PRESSURE

(75) Inventors: Dirk Freund, Kelkheim (DE); Martin Giersiepen, Oberursel (DE); Brigitte Harttmann, Niedernhausen (DE); Ulrich Heck, Krefeld (DE); Stefan Hollinger, Kronberg (DE); Frank Kressmann, Eschborn (DE); Gerrit Roenneberg, Darmstadt (DE); Fred Schnak, Kronberg (DE); Dieter Wunder, Schotten (DE)

Correspondence Address: DARBY & DARBY P.C., P.O. BOX 770, Church Street Station, New York, NY, 10008-0770, US

(73) Assignee: KAZ USA, INC., Southborough, MA (US)

(21) Appl. No.: 11/722,344

(22) PCT Filed: Dec. 15, 2005
(86) PCT No.: PCT/EP2005/013491

§ 371 Date: Sep. 11, 2007

(30) Foreign Application Priority Data
Dec. 20, 2004 (DE) 102004062435.6

(51) Int. Cl. A61B 5/026

(52) U.S. Cl. 600/504

(57) ABSTRACT

A method and a device for the non-invasive determination of blood flow and associated parameters in arteries, in particular the arterial waveform and the blood pressure. According to said method, a sensor having at least one deformable contact portion is placed on a tissue surface, essentially over an artery, and the contact portion is subjected to a temporally variable and defined external force, F(t). The deformation of the contact portion by the blood flow inside the artery in response to said force F(t) is measured.

### CROSS-REFERENCE TO PRIOR APPLICATION

This is a U.S. National Phase application under 35 U.S.C. §371 of International Application No. PCT/EP2005/013491, filed Dec. 15, 2005 and claims the benefit of German Patent Application No. 10 2004 062 435.6, filed on Dec. 20, 2004, both of which are incorporated herein. The International Application was published in German on Jun. 29, 2006 as WO 2006/066793 under PCT Article 21 (2).

### FIELD OF THE INVENTION

The invention relates to a method and a device for the non-invasive determination of blood flow and associated parameters in arteries, in particular the arterial waveform and the blood pressure. According to said method, a sensor comprising at least one deformable contact portion is placed on a tissue surface, essentially over an artery, and the at least one contact portion is subjected to a temporally variable and defined external force, F(t).

The invention also relates to a device for non-invasive determination of blood flow and associated parameters in arteries, in particular the arterial waveform and the blood pressure. The determination is accomplished with a sensor which has at least one deformable contact portion which can be subjected to a temporally variable and defined external force, F(t).

### BACKGROUND

In the customary method of measuring blood pressure employing an oscillometric measuring device, the amplitudes of pressure oscillations in an air cuff device are determined. The air cuff sleeve is applied to the measuring locus, and the pressure on the artery is increased. The artery then pulses against the pressure of the cuff. The pulsations can be detected in the cuff cavity in the form of oscillations of the interior pressure. The mean arterial pressure (MAP), systolic pressure (maximum arterial pressure), and diastolic pressure (minimum arterial pressure) can be determined from the characteristic envelope curve (amplitude of the pressure oscillation as a function of sleeve pressure). When such oscillometric devices are used, the upper arm or wrist of the patient is radially compressed by the cuff pressure until the blood flow in the arteries is stopped.

This type of arrangement is not capable of determining the pulse wave in the artery by evaluating the pressure in the cuff in a manner which accurately reflects amplitude and phase, because the arterial pulse wave is rendered inaccurately as a result of various factors. Chief among these factors are the properties of the materials of construction of the cuff and sleeve, in light of the compressibility of the air.

Also, the viscoelastic properties of the compressed tissue, and the accommodation of the blood under pressure in a plurality of arteries (e.g. radial and ulnar arteries, in the case of the wrist) which do not have identical contributions to the oscillation pulse curve in the cuff, distort the measurement results. Nonetheless, the true pulse waveform in the artery does contain important information about the circulatory system and the heart. The principal means of determining these waveforms under the state of the art, however, are invasive, namely introduction of a catheter into various arteries. The cardiologist then obtains physiological parameters of the cardiovascular system from the waveforms determined.

U.S. Pat. No. 5,450,852 discloses a method and device of the general type described supra, for determining the true pulse waveform in an artery. The sensor described in that disclosure, in particular the deformable contact portion of said sensor, is fixed over the artery, e.g. on the wrist, and the pressure inside the sensor is then increased. The pulsing of the artery against the thus increased pressure causes pressure oscillations in the sensor which oscillations can be measured with a pressure-measuring device, from which measurements the pulse waveform can be derived. However, it is necessary that this sensor be positioned relatively accurately above the artery. In general, medically trained personnel are required to achieve this correct positioning. In addition, the pressure oscillations can be rendered inaccurately as a result of various factors, e.g. properties of the material of construction of the sensor (which can increase or decrease the pressure oscillations).

DE 69720274 T2 discloses a method and device for measuring blood pressure based on evaluation of pulse wave information. Thus this publication does not directly concern oscillometric blood pressure measurement. A disadvantage of the approach disclosed in this reference is that the pressure is applied to the sensor from the exterior. As a result, the pressure applied to the sensor is less accurately controllable; also, the equipment cost is relatively high.

### SUMMARY OF THE INVENTION

Accordingly, the underlying problem of the invention was to devise a method and a device which enables measurement of blood flow and associated parameters in arteries to be accomplished by persons other than trained personnel, without inaccuracies in the reactions of the arteries to the applied force.

In the present invention, the deformation of the at least one contact portion via the blood flow in the artery as a reaction to the application of the force F(t) is measured. Because the deformation of the contact portion applied directly to the tissue surface is measured, and thus the deformation of the contact portion accurately reflects the deformation of the tissue surface by the arterial pressure, a much more sensitive determination of the blood flow and associated parameters in the arteries is enabled.

The force F(t) can advantageously be generated by increasing the pressure inside the sensor. This can be accomplished without expensive equipment, e.g. it can be accomplished via a pump which applies pressure to a fluid which fluid is accommodated inside the sensor. This pressure is well measurable, being linearly related to the force over the surface of the at least one contact portion. According to a first advantageous refinement of the invention, the deformation of a plurality of contact portions arranged in the form of an array or matrix is measured. This arrangement substantially facilitates the operation of the device, particularly for nonmedical lay persons. Because a plurality of contact portions are employed, it is unnecessary to achieve exact placement of the device on the tissue surface over an artery. Rather, it suffices if one (or some) of the contact portions is/are positioned so as to be able to measure the reaction of the artery to the application of the force F(t).

The force F(t) may be applied manually, e.g. via a spring mechanism, to the at least one contact portion.

Alternatively, force F(t) may be applied to the contact portion by an electrical motor or pneumatic device.

It has been found to be advantageous if the force F(t) is increased at a constant rate over time, e.g. in the form of a linear ramp function R(t)=t. With such a linear force function for the force applied to the tissue, one is assured of a simple correlation between the deformation of the at least one contact portion and the force F(t) introduced into the tissue.

Advantageously, the deformation of the at least one contact portion is determined via the curvature ε(t) of said portion (portions).

According to another advantageous refinement of the invention, the determination is carried out at an upper arm, wrist, temporal region, ankle, or finger of the individual. At these locations on the body, an artery is present at a relatively shallow depth below the tissue surface, whereby the signal obtained is strong and easily evaluable, and particularly well suited for purposes of the evaluation.

The underlying problem concerning the device is solved with a device comprised of a sensor which has a measuring device by which the deformation of the at least one contact portion can be determined via the blood flow in the artery as a reaction to the application of the force F(t). The deformation of the tissue surface above the artery through which blood is flowing is transmitted directly to the at least one contact portion. There is no loss of information such as is suffered according to the state of the art; under the state of the art the deformation of the contact portion is not measured directly but rather indirectly, namely via the variation of the pressure inside the sensor.

It is particularly advantageous if the forcing organ is in the form of a pneumatic unit, e.g. a fluid pump, whereby one can control the interior pressure in the sensor chamber and thus the force acting on the contact portion (or portions).

According to a refinement of the invention, the contact portion is in the form of a membrane. Such membranes are thin and flexible, ensuring that only the force F(t) and the deformation of the tissue surface via the arterial pressure as a reaction to the force F(t) are transmitted to the membrane, and ensuring that transverse forces can be ignored.

According to another advantageous refinement of the invention, the sensor has a chamber which can be filled with a fluid, which chamber has generally rigid non-deformable walls which however have at least one contact portion which is deformable. Because the force can be exerted on the contact portion (portions) (or membrane) via the fluid, a defined measurable external force can be applied to the tissue above the artery, and the reaction of the artery to this force can be measured. Advantageously, the fluid appropriately attenuates (dampens) the deformation of the contact portion (or membrane) in the measurement of the reaction of the artery to this force, so that excessive oscillation of the contact portion is avoided or is negligible.

It is particularly advantageous if a plurality of contact portions arranged in the form of an array or matrix are provided, wherewith a separate measuring device is provided for each such contact portion. Here again, this feature substantially facilitates the operation of the device, particularly for persons who are not medically trained. It is sufficient if the force is introduced into the tissue above the artery via at least one of the contact portions, and if one (or some) of the contact portions is/are positioned so as to be able to measure the reaction of the artery to the application of this applied force. It is unnecessary to achieve exact placement of the sensor.

The measurement of the deformation of the contact portion (or membrane) may be accomplished in a simple and convenient manner if each measuring device comprises a strain-measuring strip which is readily capable of determining the deformation (or curvature ε(t)) of the membrane at any point.

According to another advantageous refinement of the invention, a forcing organ is provided to exert the force. The forcing organ itself may have means of measuring the force F(t); or alternatively a separate force-measuring device may be provided.

According to a refinement of the invention, the forcing organ may be in the form of a mechanical, manually operated unit by which the force F(t) is generated, e.g. via a manually operable spring mechanism.

Alternatively, the forcing organ may be in the form of an electromechanical motor-driven unit.

It has been found to be advantageous if he measuring device for measuring the force is in the form of a pressure-measuring device which measures the fluid pressure inside the chamber, which is linearly correlated with the said force.

In order to be able to readily make use of the reaction of the artery to the force introduced into the tissue, namely to make use of it for determining the arterial waveform, it is proposed that the force F(t) be increased at a constant rate over time, e.g. in the form of a linear ramp function R(t)=λt.

According to a particularly advantageous refinement of the invention, a holding element is provided, by which the device can be fixed to, e.g., a wrist, upper arm, or finger, of the user, so as to interact with (“detect”) an artery located near the surface of the tissue.

Additional objectives, advantages, features, and potential applications of the invention will be apparent from the following description of an exemplary embodiment, with reference to the Figure.

### BRIEF DESCRIPTION OF THE DRAWINGS

FIG. 1 shows a cross sectional view of an embodiment of the inventive device for non-invasive determination of blood flow and associated parameters in arteries.

### DETAILED DESCRIPTION

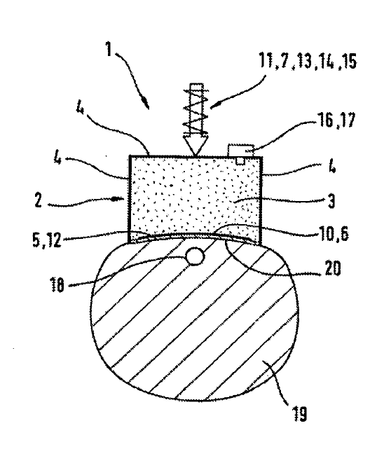

The device shown in FIG. 1 has a sensor 1 comprised essentially of a non-deformable rigid chamber 2; thus the device illustrated comprises a single-chamber sensor. The chamber 2 is filled with a fluid (gas or liquid) 3, and has rigid walls 4 and a non-rigid contact portion 5. The contact portion 5, which is intended to be placed on or against the tissue surface 20 of the user is in the form of a deformable elastic membrane 12. The membrane 12 bears a strain-measurement strip 10 which enables determination of the curvature ε(t), and thus the deformation, of the membrane 12 at any point or at certain points.

Preferably an array or matrix, particularly a two-dimensional flat “measuring field” comprised of a plurality of strain-measurement strips, is provided at the contact portion of the sensor. With this arrangement it is of less importance to achieve the optimal positioning with respect to the optimal measuring location on the artery, because one has improved probability that at least one of the strain-measurement strips is optimally disposed within the measurement field.

It is also possible to compare the different signals of the individual strain-measurement strips and thereby to carry out a validation test of the individual signals. Further, the creation of an areal measurement field enables determination of other cardiologic parameters, e.g. the propagation speed of the pressure wave. Also, calibration can be performed at the same locus on the tissue at which the arterial pressure wave is sensed.

For determination of the blood flow and associated parameters, the membrane 12 is subjected to a force F(t). Based on the reaction of the artery 18 (in which blood is flowing) to the force F(t), the desired relationships to the blood flow and associated parameters can be ascertained.

To apply the force F(t) to the membrane 12, a forcing organ 11 is provided. In the exemplary embodiment illustrated, the forcing organ is a fluid pump 7 (shown only schematically in FIG. 1) whereby a fluid 3 can be pumped into the chamber 2. The force exerted on the contact portion 5 and membrane 12 depends on the amount of fluid in the chamber 2.

A force-measuring device 17 is provided, to measure the force acting on the membrane 12. In the exemplary embodiment illustrated, in which the force is produced by increasing the pressure, the force-measuring device is a pressure-measuring device.

Alternatively to the fluid pump 7, the means of exertion of the force may be manual, via a mechanical unit 13, e.g. a manually actuated (or manually driven) spring mechanism. Another alternative is to provide the forcing organ 11 in the form of an electromechanical motor-driven unit 14 or a displacement unit, which exerts a defined force on the rigid wall opposite to the deformable wall component bearing the contact portion. In this embodiment the entire sensor is pressed toward the artery; in a variant embodiment, the part of the wall which immediately adjoins the deformable region is also elastic, to avoid any rigid end structure being pressed against the measurement location on the patient.

For the non-invasive determination of the blood flow and associated parameters in arteries 18, the sensor 1 with its contact portion 5 is applied to the surface 20 of a tissue 19 in such a way that the contact portion comes to lie essentially over an artery 18. Then the forcing organ 11 is employed, namely, in the embodiment illustrated, to increase the pressure in the fluid chamber 2 by means of the fluid pump 7 and thereby to increase the force on the contact portion 5 and the membrane 12. The force F(t) can be increased in a linear ramp function form R(t)=t, where is a constant. This temporally variable force F(t) is measured by means of the force-measuring device 17, which in this instance is a pressure-measuring device 16.

The force F(t) acting on the membrane 12 is transmitted to the tissue surface 20 and into the interior of the tissue 19 to the artery 18. The pulsing blood flow in the artery 18 mediates the reaction of the artery to the received force F(t). This reaction exerts a counterforce on the tissue 19 (and thereby on the tissue surface 20 and the membrane 12). As a result of the reaction of the artery 18 to the applied force F(t), the membrane 12 is deformed, i.e. its curvature ε(t) is changed, as a function of time. This curvature ε(t) is determinable by means of the strain-measuring strips 20.

This temporally varying curvature ε(t) deriving from the applied force F(t) and the reaction of the artery 18 to the force is characteristic for the blood flow and associated parameters in the artery, particularly
the arterial waveform and cardiovascular parameters associated with said waveform, and
the blood pressure;
   accordingly, with the aid of an appropriate evaluation procedure and appropriate evaluation instrumentation, these values can be obtained from the curvature ε(t).

The device is comprised of:
a fluid-filled single-chamber sensor, with the external force being determined via a pressure sensor for measurement of the interior fluid pressure in the chamber, or via a force sensor for measurement of the externally applied force;
a deformable membrane disposed at the contact portion between the tissue (e.g. skin tissue) and the sensor;
adjoining said membrane, inside the chamber, measuring devices for curvature and/or strain,
   which devices form a measurement field, which devices serve to determine the temporally varying curvature (and/or strain) of the membrane caused by the arterial pulsation.

1. A method for the non-invasive determination of blood flow and associated parameters in arteries comprising:
placing a sensor including at least one deformable contact portion on a tissue surface, the at least one contact portion being subjected to a temporally variable and defined external force, F(t);
wherein deformation of the at least one contact portion resulting from blood flow in the artery as a reaction to the application of the force F(t) is measured; and
wherein the force F(t) is generated by pressure increase inside the sensor itself.

2. A method according to claim 1 wherein the deformation of a plurality of contact portions arranged in the form of an array is measured.

3. A method according to claim 1 wherein the force F(t) is applied manually, to the at least one contact portion.

4. A method according to claim 1 wherein the force F(t) is applied to the contact portion by an electrical motor or pneumatic device.

5. A method according to claim 1 wherein the force F(t) is increased at a constant rate over time in the form of a linear ramp function R(t)=λt.

6. A method according to claim 1 wherein the deformation of the at least one contact portion is measurable via the curvature ε(t) of the contact portion.

7. A method according to claim 1 wherein the determination is carried out at an upper arm, wrist, temporal region, ankle, or finger of a user.

8. A device for the non-invasive determination of blood flow and associated parameters in arteries comprising:
a sensor having at least one deformable contact portion operable to be subjected to a temporally variable and defined external force, F(t);
wherein the sensor has a measuring device operable to determine deformation of the at least one contact portion via the blood flow in the artery as a reaction to the application of the force F(t); and
wherein the sensor is in the form of a single-chamber sensor; and
wherein the forcing organ is in the form of a pneumatic unit.

9. A device according to claim 8 wherein the sensor has a chamber filled with a fluid the chamber having generally rigid non-deformable walls and the at least one contact portion which is deformable.

10. A device according to claim 8, wherein the at least one deformable contact portion is in the form of a membrane.

11. A device according to claim 8, wherein a plurality of contact portions arranged in the form of an array or matrix are provided, and a separate measuring device is provided for each such contact portion.

12. A device according to claim 8, wherein the measuring device comprises a strain-measuring strip.

13. A device according to claim 8, wherein a forcing organ is provided to exert the force.

14. A device according to claim 13, wherein the forcing organ has means for measuring the force F(t).

15. A device according to claim 13, wherein a separate force-measuring device is provided.

16. A device according to claim 8, wherein the pneumatic unit comprises a fluid pump.

17. A device according to claim 12, wherein the measuring device is in the form of a pressure-measuring device for measuring the fluid pressure inside the chamber.

18. A device according to claim 8, wherein the force F(t) is increased at a constant rate over time, in the form of a linear ramp function R(t)=λt.

19. A device according to claim 8, further comprising a holding element, by which the device is fixable to a wrist, upper arm, temple area, ankle, or finger, of a user.
